(19) United States
(12) Patent Application Publication
(10) Pub. No.: US 2015/0038927 A1
(43) Pub. Date: Feb. 5, 2015

(54) Surgical Drain Reservoir Support

(71) Applicant: Mary Ann Robinson Prody, White Bear Lake, MN (US)

(72) Inventor: Mary Ann Robinson Prody, White Bear Lake, MN (US)

(21) Appl. No.: 14/450,260

(22) Filed: Aug. 3, 2014

Related U.S. Application Data
(60) Provisional application No. 61/958,662, filed on Aug. 3, 2013.

(51) Int. Cl. A61F 5/44; A61M 27/00

(52) U.S. Cl. CPC A61F 5/4408; A61M 27/00
USPC 604/327

(57) ABSTRACT

The Improved Surgical Drain Reservoir Support is a system to support/secure surgical drainage reservoirs.

### CROSS REFERENCE TO RELATED APPLICATIONS

The application listed below is the only application related to this application. This application claims benefit of the earlier filing date under 35 USC 119(e) of provisional application No. 61/958,662 filed on Aug. 3, 2013.

### STATEMENT REGARDING FEDERALLY SPONSORED RESEARCH OR DEVELOPMENT

No invention claimed in this application was made under Federally sponsored research or development.

### THE NAMES OF THE PARTIES TO A JOINT RESEARCH AGREEMENT

Not Applicable

### REFERENCE TO A SEQUENCE LISTING

Not Applicable

### BACKGROUND OF THE INVENTION

1. Field of the Invention

This invention relates to an improvement of the securement of surgical drainage reservoirs, more particularly a belt that provides one or more fasteners to secure the drainage reservoirs within the sight and reach of the patient. Certain surgical procedures require surgical placement of a drain tube into the body at the surgical site to remove drainage and promote surgical healing. The drain tube is connected to a drainage reservoir that accumulates drainage, which is outside the body. The drainage reservoir is then emptied regularly, often several times a day, by clinicians and then patients are taught how to empty drainage reservoirs.

It is an object of the present invention to provide an improved securement of surgical drainage reservoirs while improving the ease with which clinicians and patients can use this device. This improvement provides a handle, or gripping surface, to the previously described fastener. The handle allows the clinician or patient easier opening of the fastener, which by description of hook and loop closure is so secure it can present a challenge when removing and replacing drainage reservoirs for emptying.

2. Description of Related Art

Supports for surgical drainage reservoirs whereby one or more drainage reservoirs 2 is easily and securely fastened to a band within sight and reach of the patient, such as the device described in U.S. Pat. No. 6,610,032 are very useful and prevent complications for post surgical drainage from surgical sites requiring drainage devices. What is needed is a modification to a support for surgical drainage devices that allows patients with altered or limited dexterity such as patients with arthritis or other disabilities, and/or patients with diminished strength to more easily access and use the described fasteners.

### BRIEF SUMMARY OF THE INVENTION

The Improved Surgical Drain Reservoir Support is a system to support/secure surgical drainage reservoirs. The support system is a band, preferably a soft flexible, elastic fabric such as cotton/polyester that stretches for adjustment to fit a range of animal, particularly vertebrate animal, body part sizes. The band is placed about a body part of a patient, at a selected tightness, the fastening means being adjustable as to said tightness. The band is fastened at the front. Attached to the outside surface of the band preferably there would be one or more spaced apart elongate fasteners aligned within a longitudinal axis of the band.

### BRIEF DESCRIPTION OF THE SEVERAL VIEWS OF THE DRAWING

FIG. 1 is a perspective view of an embodiment of the invention.

FIG. 2 is a perspective view of a second embodiment of the invention.

### DETAILED DESCRIPTION OF THE INVENTION

The subject of this patent application is a fabric system 1 to support/secure surgical drainage reservoirs 2. The support system is a fabric band 3, preferably a soft flexible, elastic fabric such as cotton/polyester that stretches for adjustment to fit a range of vertebrate, and invertebrate body part sizes. The band 3 is placed about a body part of a patient, at a selected tightness, the fastening means being adjustable as to said tightness. The patient may be any human, vertebrate animal, or invertebrate animal having suitable body parts. The band 3 is fastened at the front by engaging the inside of one end with a hooking structure to the outside of the other end to a looping structure which is adapted for mutual engagement through contact.

Attached to the outside surface of the band 3 preferably there would be a plurality of spaced apart elongate fasteners 4 aligned within a longitudinal axis of the band 3. Each of the fasteners preferably compromising a first surface attachment element integral with the outside surface of the band 3, and a second surface attachment element permanently fastened, as in sewing, at one end of the first surface attachment element.

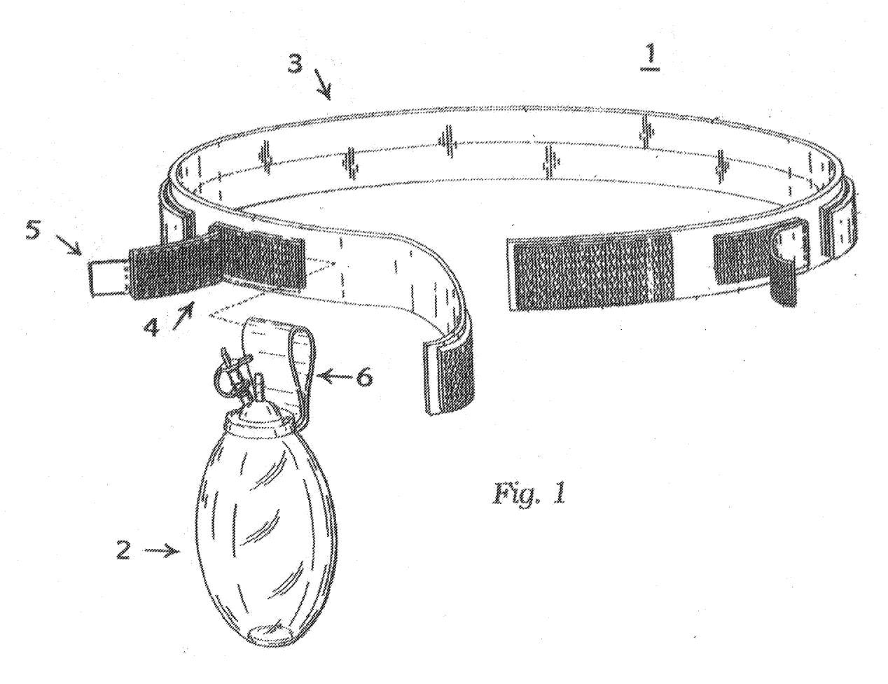

On each of the fasteners, on the end that is not permanently fastened to the band 3, there would be a handle 5, preferably fabric, or loops, molded plastic or metal on the second surface attachment element that is permanently attached, as in sewing or other method, including, without limitation, rivets, staples, or other attachment, that extends outwardly past the second surface attachment element in less, same or more width than the second surface attachment element which would provide an extended surface. The extended surface would provide a surface so a person may easily grip, as in to open, the elongate fastener 4, separating the second surface attachment element from the first surface attachment element. (FIG. 1).

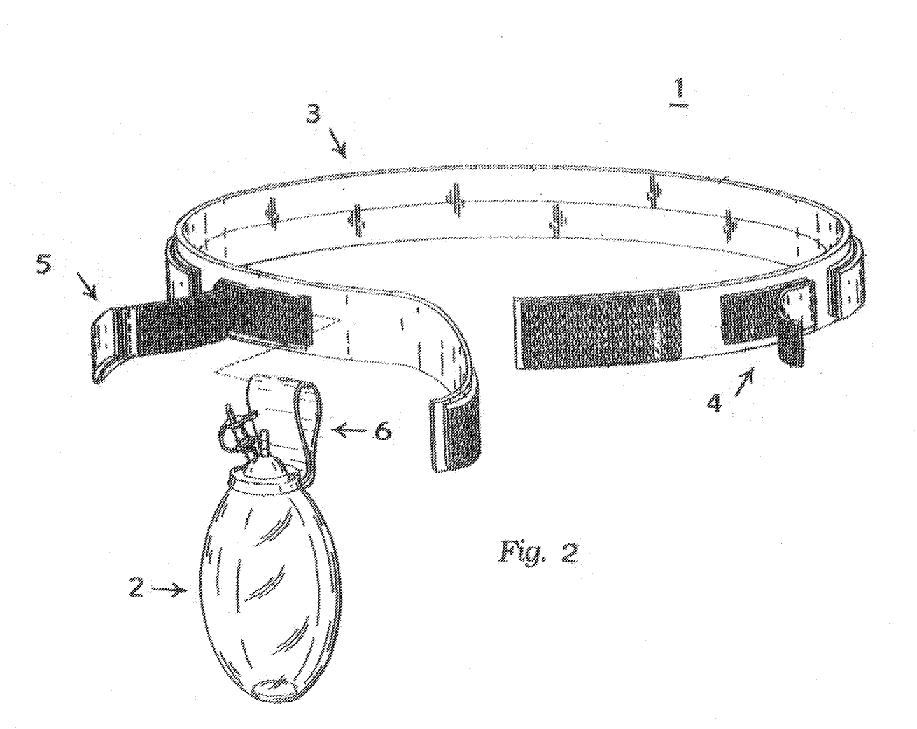

In another embodiment, on each fastener on the end that is not permanently fastened, on the second surface attachment element there would be a handle 5 comprised of an extension of the second surface attachment element. In one embodiment, the extension would be comprised of the same hook/loop material where in the outside surface is permanently fastened to the inside surface, as by sewing, extending outwardly past the second surface attachment in the same width of the second surface attachment element which would provide a handle 5 so a person may easily grip as in to open the elongate fastener separating the second surface attachment element from the first surface attachment element. (FIG. 2)

In operation, a patient would apply, or have applied for them, the fabric band 3 around a vertebrate body part and secured at a selected tightness, the fastening means being adjustable as to said tightness. The patient may adjust the size for a comfortable and secure fit, or have it so adjusted for them. In function, the fabric band 3 will support/secure surgical drainage reservoirs 2 extending from a patient's body. Surgical tube extending from a patient's body with an attached drainage reservoir would be supported/secured by gripping the less, same or more width attachment element of the second surface attachment element of one of the elongate fasteners which is then manually pulled away from its companion first attachment element so that it is free, opens outwardly and is threaded through the loop 6 on the surgical drainage device and supported/secured by adhering the hook part to the loop 6 part of the Velcro-type fastener.

In another embodiment, the surgical drainage reservoir 2 would be supported/secured by gripping the extended surface of the same width attachment element of the second surface attachment element of one of the elongate fasteners which is then manually pulled away from its companion first attachment element so that it is free, opens outwardly and is threaded through the loop on the surgical drainage device and supported/secured by adhering the hook part to the loop part of the Velcro-type fastener. This procedure may be followed for attaching additional surgical drainage devices to the other elongate fasteners so as to mount the surgical drainage devices within visual range and reach of the patient.

A patient may have one or more surgical drainage reservoirs at one time extending from the patient's body, which would allow the patient to utilize the Velcro-type fasteners on either sides of the fabric band 3 corresponding with the location of the surgical drainage devices extending from the patient's body.

I claim:

1. An apparatus for collecting a bodily fluid in a medical draining procedure, the apparatus comprising:
a) a belt adapted for extending about the body part of a patient including a belt fastening means for securing the belt at a selected tightness thereon, the securing means being adjustable as to said tightness;
b) an outside surface of the belt providing one or more spaced apart elongate fasteners aligned with a longitudinal axis of the belt, each of the fasteners comprising a first surface attachment element integral with the outside surface of the belt, and a second surface attachment element permanently fastened at one end of the first surface attachment element and extending longitudinally in removable, mutually engaged contact with the first surface attachment element, said second surface attachment element further comprising a handle;
c) a drainage reservoir providing an attachment band comprising a loop adapted for receiving one of said second surface attachment element for engaging the drainage reservoir with the belt for disposing the drainage reservoir within reach of a person wearing the belt.

2. The apparatus of claim 1 wherein the belt is of an elastic material for stretch adjustment to fit a range of body part sizes.

3. The apparatus of claim 1 wherein the drainage reservoir is a flexible bulb enabled for expulsion of the fluid stored therein by compressing the bulb.

4. The apparatus of claim 1 wherein the drainage reservoir is of a transparent material so as to enable the individual to see when the drainage reservoir is filled.

5. The apparatus of claim 4 wherein the plural spaced apart elongate fasteners comprise at least one such fastener on each side of the belt fastening means, the elongate fasteners placed frontally so as to be within normal visual access of the individual.

6. The apparatus of claim 1 wherein the first surface attachment element comprises a surface of outwardly directed hooking structures and the second surface attachment element comprises a surface of outwardly directed looping structures, wherein the hooking structures and the looping structures are adapted for mutual engagement through contact.

7. A device for supporting one or more drainage reservoirs attached to a patient, each drainage reservoir having an aperture associated therewith for supporting the drainage reservoir, the device comprising:
a) a belt to be attached about the body part of the patient; and
b) one or more fasteners attached to the belt, wherein each fastener is for removably attaching one or more drainage reservoirs to the belt and includes a first end permanently attached to the belt and a second end adapted to releasably engage the belt, each fastener adapted to removably attach a drainage reservoir to the belt by threading said second end through said aperture and engaging said second end to the belt, one or more of said fasteners further comprising a handle.

8. The device of claim 7, wherein each fastener is substantially aligned with a longitudinal axis of the belt.

9. The device of claim 7, wherein the second end of each fastener includes one of a hook and loop fastener to releasably engage the belt.

10. The device of claim 7, wherein the aperture is a loop attached to one of the drainage reservoirs.

11. The device of claim 9, wherein the belt includes a plurality of attachment elements respectively associated with each fastener, each attachment element being integral with the belt to releasably engage the second end of the fastener associated therewith.

12. The device of claim 11, wherein each attachment element is a loop surface sewn to the belt.

13. The device of claim 9, wherein the second end of each fastener includes a hook surface for engaging a loop surface on the belt.

14. The device of claim 7, wherein the first end is sewn to the belt.

15. The device for supporting one or more drainage reservoirs of claim 7 wherein said fastener further comprises one or more handles.
